(19) United States
(12) Patent Application Publication
(10) Pub. No.: US 2026/0337539 A1
(43) Pub. Date: Oct. 29, 2026

(54) TRANSFORMER BIOREACTOR

(71) Applicant: ARIZONA BOARD OF REGENTS ON BEHALF OF THE UNIVERSITY OF ARIZONA, Tucson, AZ (US)

(72) Inventors: Joel L. Cuello, Tucson, AZ (US); Jack Welchert, Tucson, AZ (US)

(21) Appl. No.: 19/104,833

(22) PCT Filed: Aug. 24, 2023
(86) PCT No.: PCT/US23/72838

§ 371 Date: Feb. 19, 2025

Related U.S. Application Data
(60) Provisional application No. 63/373,443, filed on Aug. 24, 2022.

(51) Int. Cl. C12M 1/06

(52) U.S. Cl. CPC C12M 27/02

(57) ABSTRACT

A transformer bioreactor (T-bioreactor) comprising a chamber, an impeller disposed within the chamber comprising one or more blades, an agitator disposed within the chamber operatively coupled to the impeller, and an actuator operatively coupled to the agitator capable of actuating the agitator. The impeller and the one or more blades may be capable of moving into a plurality of alternate configurations by movement of a plurality of retractable cards disposed within the one or more blades. The agitator, when actuated, may cause the impeller, the one or more blades, or a combination thereof to move into an alternate configuration of the plurality of alternate configurations.

### CROSS-REFERENCES TO RELATED APPLICATIONS

This application claims benefit of U.S. Provisional Application No. 63/373,443 filed Aug. 24, 2022, the specification of which is incorporated herein in its entirety by reference.

### FIELD OF THE INVENTION

The present invention is directed to bioreactor impellers capable of dynamic, real-time adjustment of angle, shape, and configuration through the use of a remote actuator.

### BACKGROUND OF THE INVENTION

Modern bioreactors (also known as fermenters) have been around now for over a hundred years, but the design and operation of conventional bioreactors remain for the most part unchanged.

The Continuous Stirred Tank Reactor (CSTR) (FIG. 1), for instance, has its design determined by the type of agitator (also known as stirring blade or impeller) used to mix the liquid in the bioreactor (FIG. 2). Once selected, the specific agitator together with the CSTR's baffles sets and fixes the general pattern of hydrodynamic mixing within the bioreactor (FIG. 3).

With the given type and number of agitator(s) and baffles (FIG. 1) in a CSTR fixed, the specifics of the CSTR's mixing regime may only be modified by varying the agitator's speed. Thus, altering the hydrodynamic conditions within the bioreactor with respect to time to fully optimize the growth and productivity of the cell culture in the bioreactor according to the cells' varying growth stages and densities is significantly limited.

### BRIEF SUMMARY OF THE INVENTION

It is an objective of the present invention to provide systems that allow for bioreactor impellers capable of dynamic, real-time adjustment of angle, shape, and configuration through use of a remote actuator, as specified in the independent claims. Embodiments of the invention are given in the dependent claims. Embodiments of the present invention can be freely combined with each other if they are not mutually exclusive.

The present invention features a transformer bioreactor (T-bioreactor) comprising a chamber, an impeller disposed within the chamber comprising one or more blades, an agitator disposed within the chamber operatively coupled to the impeller, and an actuator operatively coupled to the agitator capable of actuating the agitator. In some embodiments, the impeller and the one or more blades may be capable of moving into a plurality of alternate configurations. In some embodiments, the agitator, when actuated, may cause the impeller, the one or more blades, or a combination thereof to move into an alternate configuration of the plurality of alternate configurations.

One of the unique and inventive technical features of the present invention is the implementation of components disposed within a bioreactor chamber, configured to change their angle or configuration upon actuation. Without wishing to limit the invention to any theory or mechanism, it is believed that the technical feature of the present invention advantageously provides for efficient and remote shifting of a shape, size, angle, or hole pattern of the components, allowing for the fluid flow within the chamber to be altered from the exterior without needing to swap out components. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of a CSTR illustrating the side baffles.

FIG. 2 shows types of agitator or impeller used in a CSTR.

FIG. 3 shows types of flow regimes generated by radial and axial impellers in a CSTR.

FIG. 4 shows a schematic for a general mechanical design of a T-Impeller whose angular orientations may be dynamically altered.

FIG. 5 shows a schematic for a general mechanical design of a T-Impeller consisting of a set of retractable T-Cards whose surface may either be solid or with patterns of holes of varying size, geometric shape, and distribution pattern. Not drawn to scale and with proper dimensional proportions; with an exaggerated width to highlight the T-Cards.

FIG. 6 shows a schematic for a general mechanical design of a T-Impeller consisting of a set of retractable T-Cards, operated with no T-Card drawn out.

FIG. 7 shows a schematic for a general mechanical design of a T-Impeller consisting of a set of retractable T-Cards, operated with a solid T-Card drawn out.

FIG. 8 shows a schematic for a general mechanical design of a T-Impeller consisting of a set of retractable T-Cards, operated with a T-Card with large circular holes.

FIG. 9 shows a schematic for a general mechanical design of a T-Impeller consisting of a set of retractable T-Cards, operated with a T-Card with small circular holes.

FIG. 10 shows a schematic for a general mechanical design of a T-Impeller consisting of a set of retractable T-Cards, operated with a T-Card with rectangular holes.

FIG. 11 shows a schematic for a general mechanical design of a T-Impeller consisting of a set of retractable T-Cards, operated with a T-Card with rectangular holes wherein each rectangular hole may be fully or partially covered or closed.

FIGS. 12A-12D show schematics for a mechanical design of an expandable and collapsible T-Impeller with a stretchable polymer material that is either transparent or opaque.

FIGS. 13A-13E show schematics of the design of T-Bioreactor baffle (or T-Baffle) which may be dynamically altered. FIG. 13A shows an embodiment of a rectangular baffle. FIG. 13B shows a rectangular baffle with a rectangular solid card drawn out. FIG. 13C shows a rectangular baffle with a triangular solid card drawn out. FIG. 13D shows a rectangular baffle with a drawn-out rectangular card with circular holes. FIG. 13E shows a rectangular baffle with a drawn-out triangular card with circular holes.

FIG. 14 shows a schematic for a mechanical design of an expandable and collapsible T-Impeller with a stretchable transparent polymer material and equipped internally with panels of light emitting diodes (LEDs) to provide light (photosynthetically active radiation) to photosynthetic cells being cultivated, and whose light intensity may be adjusted dynamically (real-time) and remotely.

FIG. 15 shows an embodiment of the present invention with multiple impellers disposed on a single central pole.

### DETAILED DESCRIPTION OF THE INVENTION

Following is a list of elements corresponding to a particular element referred to herein:
100 bioreactor
110 chamber
120 impeller
125 blades
127 retractable cards
130 agitator
140 actuator
150 baffle
160 motor

The term “baffle” is defined herein as a device in the reactor vessel, which is a typical feature in stirred tank reactors, used to break the circulating flow pattern to prevent vortex formation and to improve overall bulk-liquid mixing.

The term “impeller” is defined herein as a driven rotor used to increase the pressure and flow of a fluid.

The term “agitator” is defined herein as a component configured to stir or disturb a component (e.g. the impeller) briskly.

The term “axial” is defined herein as pertaining to the longitudinal axis of the reactor vessel.

The term “radial” is defined herein as perpendicular to the longitudinal axis of the reactor vessel.

The term “bioreactor” is defined herein as vessels or tanks in which whole cells are cultivated or cell-free enzymes transform raw materials into biochemical products and/or less undesirable by-products.

The present invention features a transformer bioreactor (T-bioreactor) (100) comprising a chamber (110), an impeller (120) disposed within the chamber (110) comprising one or more blades (125), an agitator (130) disposed within the chamber (110) operatively coupled to the impeller (120), and an actuator (140) operatively coupled to the agitator (130) capable of actuating the agitator (130). In some embodiments, the impeller (120) and the one or more blades (125) may be capable of moving into a plurality of alternate configurations. In some embodiments, the agitator (130), when actuated, may cause the impeller (120), the one or more blades (125), or a combination thereof to move into an alternate configuration of the plurality of alternate configurations.

In some embodiments, the plurality of alternate configurations comprises changing an angle of the impeller (120), the one or more blades (125), or a combination thereof. In some embodiments, the plurality of alternate configurations comprises changing a geometric form of the impeller (120), the one or more blades (125), or a combination thereof.

In some embodiments, each blade of the one or more blades (125) comprises one or more retractable cards (127). Drawing out or retracting the one or more retractable cards (127) may change the shape of the blade. Each retractable card (127) of the one or more retractable cards (127) may be solid or may comprise a hole pattern.

In some embodiments, the impeller (120) is expandable and collapsible. In some embodiments, the impeller (120) may comprise a stretchable polymer material that is transparent or opaque.

In some embodiments, the T-bioreactor (100) may further comprise one or more baffles (150) disposed on an interior surface of the chamber (110), each baffle (150) having a plurality of alternate configurations. The plurality of alternate configurations of each baffle (150) may comprise a rectangular shape, an extended rectangular shape, a rectangular shape with triangular protrusions, a rectangular shape with a hole pattern, and a rectangular shape with triangular protrusions and a hole pattern, among others.

In some embodiments, the impeller (120) may further comprise one or more light emitting diodes. In some embodiments, the actuator (140) may be wirelessly coupled to the agitator (130). In some embodiments, the actuator (140) may be coupled by a wire to the agitator (130) from outside the chamber (110). In some embodiments, the bioreactor (100) may further comprise one or more additional impellers operatively coupled to the agitator (130).

In some embodiments, the agitator (130) may comprise a vibration component that, when actuated, is configured to vibrate and alter a configuration of the one or more blades (125). In some embodiments, the agitator (130) may be configured to locally agitate one or more points on the one or more blades (125) such that the one or more retractable cards (127) can be selectively drawn out or retracted. If a blade of the one or more blades (125) comprises a plurality of retractable cards, the agitator (130) may be configured to be able to draw out one or more cards of the plurality of cards, retract one or more cards of the plurality of cards, or a combination thereof, depending on user input. In some embodiments, the actuator (140) may comprise a plurality of configurations configured to allow the user to selectively operate the agitator (130) (e.g. a switch for every card in the blades such that actuating a switch causes the corresponding card to be drawn out or retract).

In some embodiments, the impeller (120) may be driven by a motor (160) operatively coupled to the impeller (120) such that the impeller is configured to rotate upon actuation of the motor (160). In some embodiments, the motor (160) may be configured to rotate the impeller on an axis parallel with the walls of the chamber (110). In some embodiments, upon rotation of the impeller (120), the one or more blades (125) may be configured to rotate on an axis perpendicular to the walls of the chamber (110). In some embodiments, the one or more blades (125) rotating on the axis perpendicular to the walls of the chamber (110) may comprise radial rotation, axial rotation, or a combination thereof.

In some embodiments, the impeller (120) may be suspended within the chamber (110) by a central pole disposed through an upper wall of the chamber (110). Thus, the motor (160) may be operatively coupled to the central pole such that the central pole is rotated by the motor (160) to rotate the impeller (120). In some embodiments, the central pole may extend past the upper wall of the chamber (110) such that the motor (160) may be coupled to the central pole externally to the chamber (110). In some embodiments, the motor (160) may be coupled to the central pole within the chamber (110).

In some embodiments, the chamber (110) may comprise a cross-section having a circular shape, a rectangular shape, a square shape, an ellipsoid shape, or any polygonal shape. In some embodiments, the upper wall of the chamber (110) may comprise a removable lid (i.e. a screw-on lid, a snap-on lid, etc.). In some embodiments, the chamber (110) may comprise a cylindrical shape, a rounded cylindrical shape, a rectangular prism shape, or any three-dimensional shape. In some embodiments, a material of the chamber (110) may comprise metal, plastic, rubber, or any other material suitable for holding a fluid.

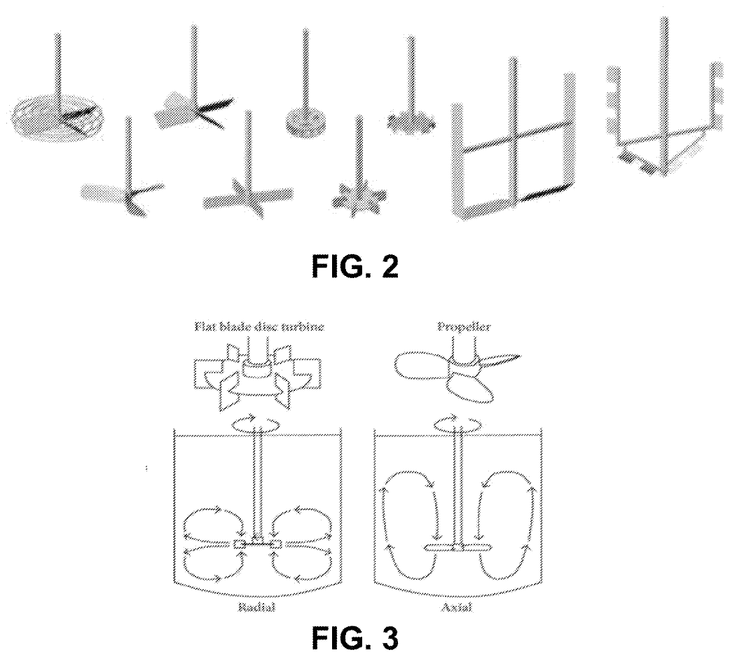

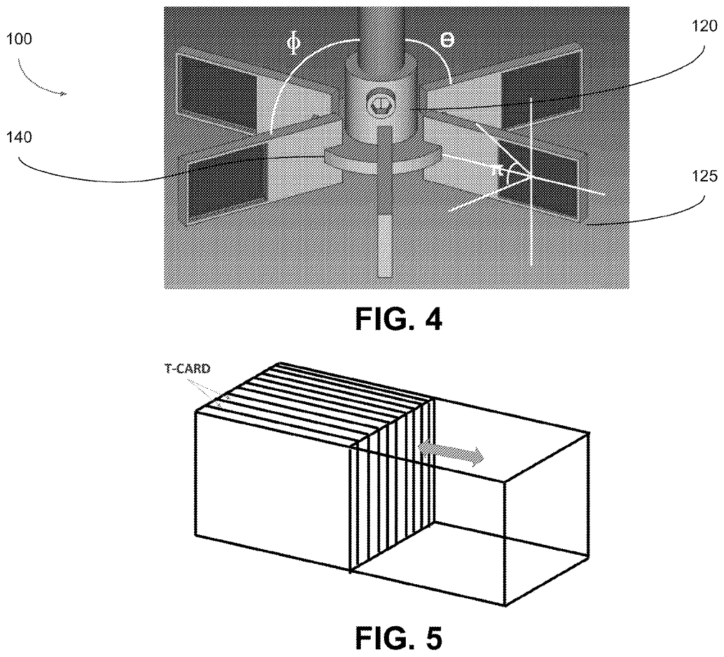

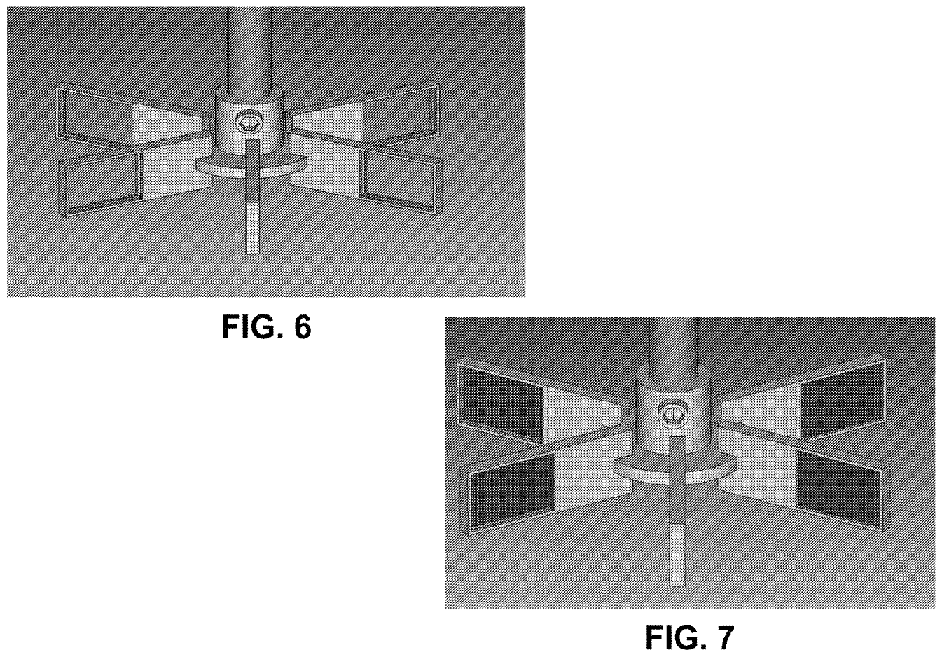

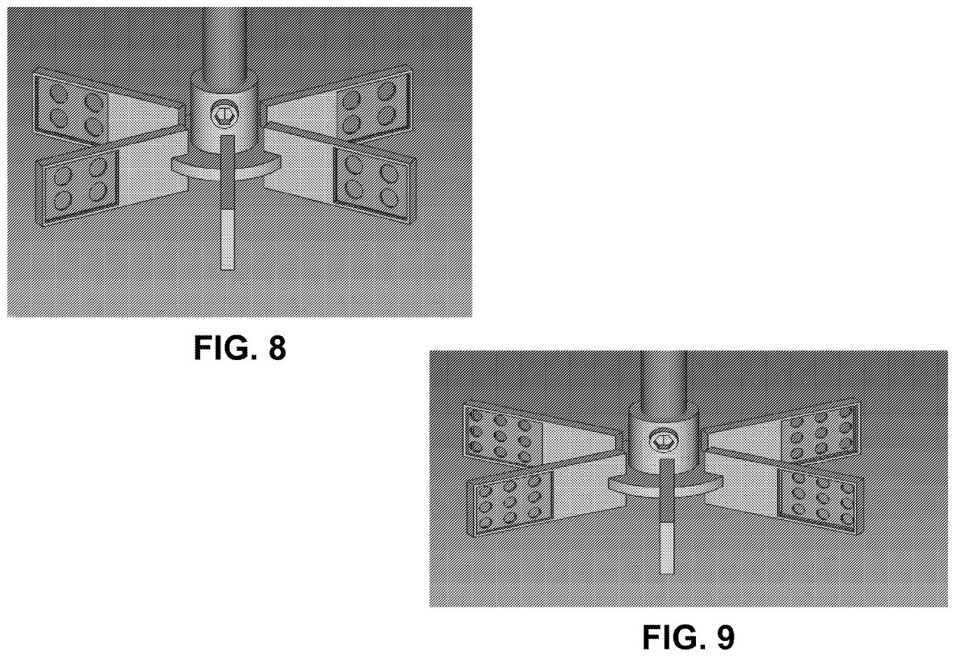

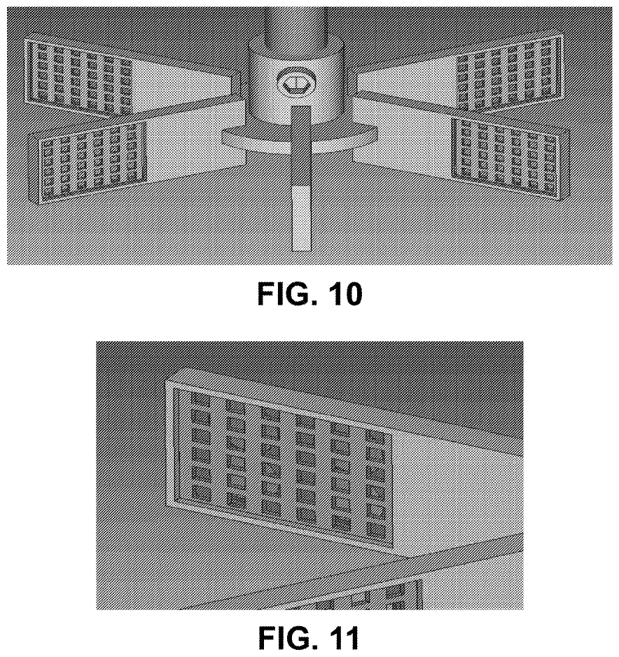

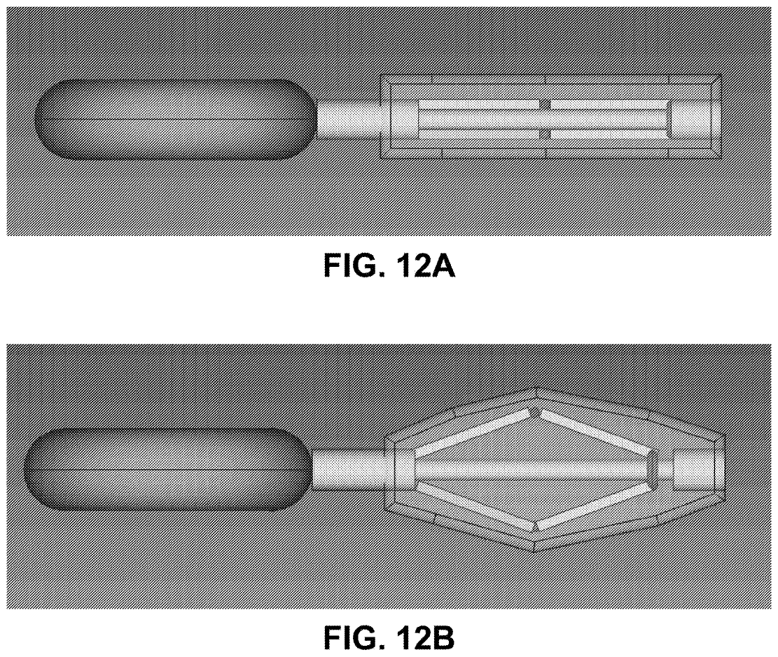

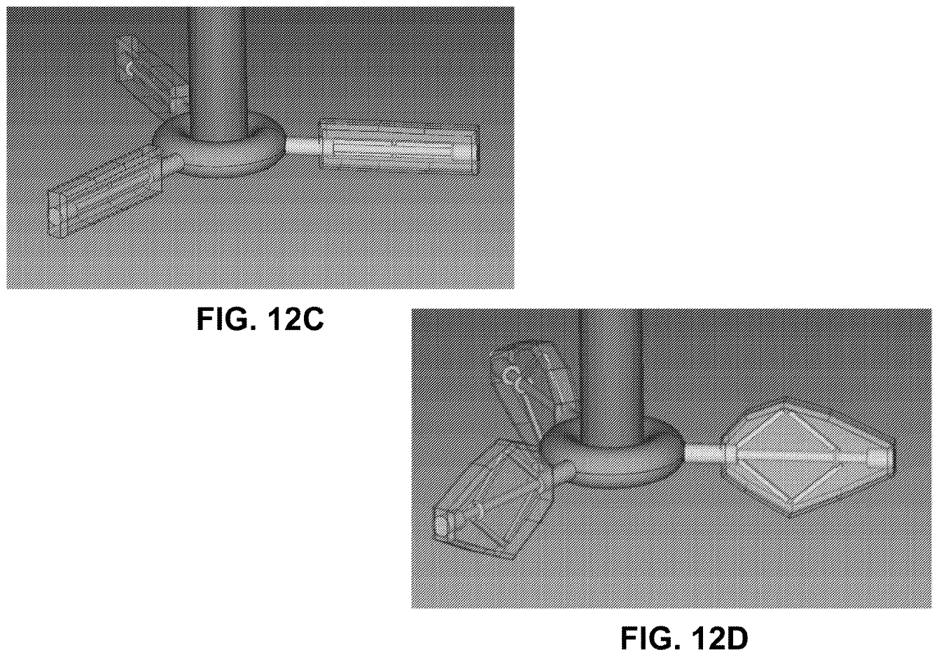

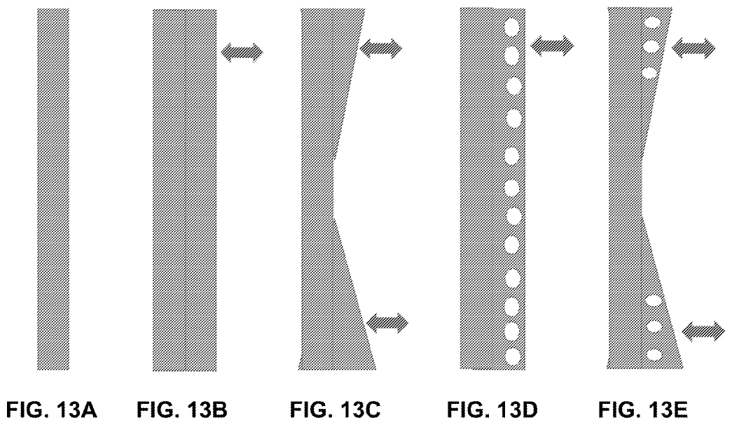

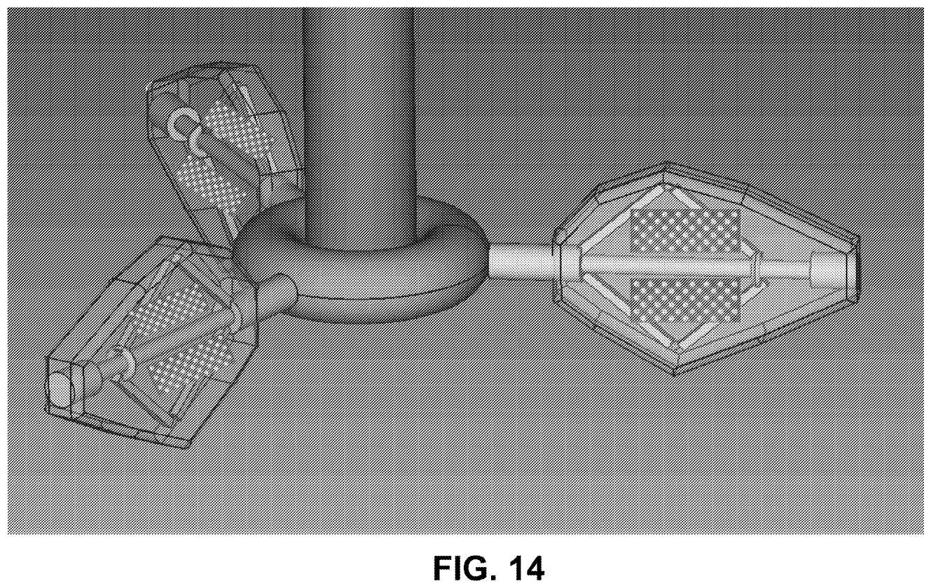

In some embodiments, the impeller (120) may comprise a plurality of rings, a plurality of rods, a plurality of discs, or a combination thereof. For example, as depicted in FIG. 2, the impeller (120) may comprise a shape similar to that of a fan wireframe comprising a disc made up of a plurality of interlocking rods.

In some embodiments, the one or more blades (125) may comprise a curved propeller blade shape. In some embodiments, at least one of the one or more blades (125) may curve in a clockwise direction. In some embodiments, at least one of the one or more blades (125) may curve in a counterclockwise direction.

In some embodiments, the one or more blades may comprise a rectangular shape. In some embodiments, the one or more blades may be angled in a radial direction, an axial direction, or a combination thereof. In some embodiments, at least one of the one or more blades (125) may be angled in a clockwise direction. In some embodiments, at least one of the one or more blades (125) may be angled in a counterclockwise direction.

In some embodiments, the one or more blades (125) may comprise one or more walls disposed above the one or more blades (125), below the one or more blades (125), or a combination thereof. The one or more walls may be configured to brace the one or more blades (125), alter a shape of the impeller (120), or a combination thereof.

In some embodiments, the impeller (120) may comprise a central disc coupled to a point on the central pole such that the one or more blades (125) extend from the central disc. In some embodiments, the central disc may be coupled to the central pole at the center of the central disc. In some embodiments, the central disc may be coupled to the central pole at any point on the central disc.

In some embodiments, all blades of the one or more blades (125) may be similar or equal in shape, size, retractable card configuration, or any other property. In other embodiments, at least one of the one or more blades (125) may differ in shape, size, retractable card configuration, or any other property from at least one other blade of the one or more blades (125).

In some embodiments, the actuator (140) may comprise a button, switch, lever, or any other component configured to generate movement of the agitator (130) such that the configuration of the one or more blades (125) may be changed. In some embodiments, the actuator (140) may be disposed on an exterior of the chamber (110). In some embodiments, the actuator (140) may be disposed at any point exterior to the chamber (110). In some embodiments, the actuator (140) may be configured to actuate the agitator (130) by a mechanical mechanism (i.e. pulleys, springs, etc.). In some embodiments, the actuator (140) may be configured to actuate the agitator (130) by an electrical mechanism.

In some embodiments, the actuator (140) may be operatively coupled to the agitator (130) by a wired connection, a wireless connection, or a combination thereof. In some embodiments, the actuator (140) may comprise a wireless transmitter and the agitator (130) may comprise a wireless receiver. In some embodiments, the wireless transmitter, the wireless receiver, or a combination thereof may comprise one or more antennas. In some embodiments, the bioreactor (100) may further comprise a power supply (e.g. a battery, a generator, a power outlet, etc.) operatively coupled to the actuator (140) such that actuating the actuator (140) causes the power supply to provide power to the agitator (130).

In some embodiments, the actuator (140) may be operatively coupled to the motor (160) such that actuating the actuator (140) causes the motor (160) to actuate and rotate the impeller (120). In some embodiments, the bioreactor (100) may further comprise a motor actuator operatively coupled to the motor (160) such that actuating the motor actuator causes the motor (160) to actuate and rotate the impeller (120). In some embodiments, the motor actuator may be operatively coupled to the power supply such that actuating the motor actuator causes the power supply to provide power to the agitator (130). In some embodiments, the actuator (140) may be operatively coupled to the one or more light-emitting diodes.

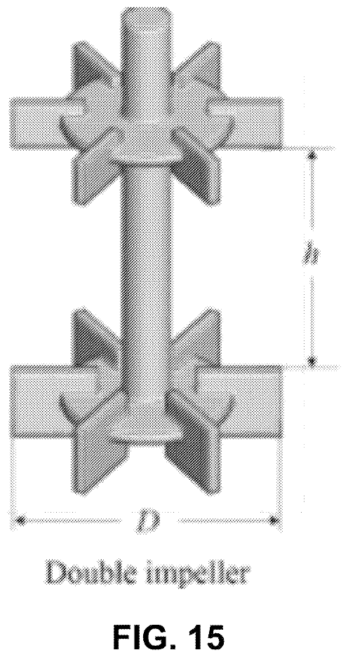

Referring to FIG. 15, a central pole may have one, two, three, or any number of impellers disposed around it. In some embodiments, a material of the one or more impellers may comprise metal polymer, or any other rigid material.

The concept of the Transformer Bioreactor (or T-Bioreactor), being introduced and claimed here, refers to any bioreactor whose physical or mechanical design of its critical component or components may be deliberately modified or varied at any time during operation—remotely, preferably—to adjust or modify the hydrodynamic conditions within the bioreactor with a view to fully optimizing the growth and/or productivity of the cells being cultivated.

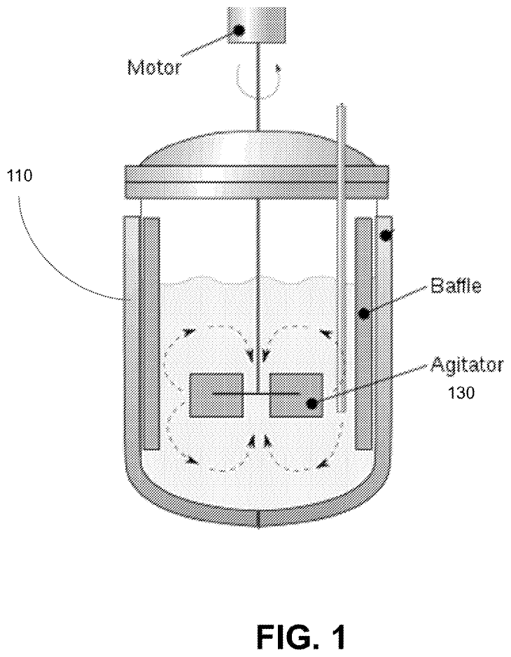

Thus, instead of equipping a CSTR with a conventional fixed or rigid agitator (FIG. 1) resulting in a set hydrodynamic pattern, the CSTR may be furnished with an agitator that during operation allows for: (1) its blade or impeller angle to be dynamically adjusted; and/or, (2) its blade or impeller mechanical design to be dynamically modified in terms of geometric form and configuration.

The foregoing impeller is referred to as a Transformer Impeller (or T-Impeller), Transformer Blade (or T-blade) or Transformer Agitator (T-Agitator), and the CSTR as a Transformer Bioreactor (or T-Bioreactor).

In some embodiments, the present invention features a transformer impeller (120) comprising one or more blades (125). The impeller (120) and the one or more blades (125) may be capable of moving into a plurality of alternate configurations upon remote actuation.

In some embodiments, the present invention features a transforming baffle (150) comprising one or more retractable cards (127). The baffle (150) and the one or more retractable cards (127) may be capable of moving into a plurality of alternate configurations upon remote actuation.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A transformer bioreactor (100) comprising:
a. a chamber (110);
b. an impeller (120) disposed within the chamber (110) comprising one or more blades (125);
c. an agitator (130) disposed within the chamber (110) operatively coupled to the impeller (120); and
d. an actuator (140) operatively coupled to the agitator (130) capable of actuating the agitator (130);
wherein the impeller (120) and the one or more blades (125) are capable of moving into a plurality of alternate configurations;
wherein the agitator (130), when actuated, causes the impeller (120), the one or more blades (125), or a combination thereof to move into an alternate configuration of the plurality of alternate configurations.

2. The bioreactor (100) of claim 1, wherein the plurality of alternate configurations comprise changing an angle of the impeller (120), the one or more blades (125), or a combination thereof.

3. The bioreactor (100) of claim 1, wherein the plurality of alternate configurations comprises changing a geometric form of the impeller (120), the one or more blades (125), or a combination thereof.

4. The bioreactor (100) of claim 1, wherein each blade of the one or more blades (125) comprises one or more retractable cards, wherein drawing out or retracting the one or more retractable cards changes a shape of the blade.

5. The bioreactor (100) of claim 4, wherein each retractable card of the one or more retractable cards is solid or comprises a hole pattern.

6. The bioreactor (100) of claim 1, wherein the impeller (120) is expandable and collapsible.

7. The bioreactor (100) of claim 6, wherein the impeller (120) comprises a stretchable polymer material.

8. The bioreactor (100) of claim 7, wherein the stretchable polymer material is transparent or opaque.

9. The bioreactor (100) of claim 1 further comprising one or more baffles disposed on an interior surface of the chamber (110), each baffle having a plurality of alternate configurations.

10. The bioreactor (100) of claim 9, wherein the plurality of alternate configurations of each baffle comprises a rectangular shape, an extended rectangular shape. a rectangular shape with triangular protrusions, a rectangular shape with a hole pattern, and a rectangular shape with triangular protrusions and a hole pattern, among others.

11. The bioreactor (100) of claim 1, wherein the impeller (120) further comprises one or more light emitting diodes.

12. The bioreactor (100) of claim 1, wherein the actuator (140) is wirelessly coupled to the agitator (130).

13. The bioreactor (100) of claim 1, wherein the actuator (140) is coupled by a wire to the agitator (130) from outside the chamber (110).

14. The bioreactor (100) of claim 1 further comprising one or more additional impellers operatively coupled to the agitator (130).

15. A transformer impeller (120) comprising one or more blades (125), wherein the impeller (120) and the one or more blades (125) are capable of moving into a plurality of alternate configurations upon remote actuation.

16. A transformer baffle comprising one or more retractable cards, wherein the baffle and the one or more retractable cards are capable of moving into a plurality of alternate configurations upon remote actuation.
